(19) United States
(12) Patent Application Publication
(10) Pub. No.: US 2014/0256924 A1
(43) Pub. Date: Sep. 11, 2014

(54) CARBOHYDRATE FUNCTIONALISED SURFACES

(71) Applicant: THE PROVOST, FELLOWS, FOUNDATION SCHOLARS, AND THE OTHER MEMBERS OF BOARD, OF THE COLLEGE OF THE HOL, Dublin 2 (IE)

(72) Inventors: Eoin M. Scanlan, Dublin (IE); Paula E. Colavita, Dublin (IE); Deirdre Murphy, Dublin (IE); Jean Bourke, Dublin (IE)

(73) Assignee: THE PROVOST, FELLOWS, FOUNDATION SCHOLARS AND THE OTHER MEMBERS OF BOARD OF, Dublin, 2 (IE)

(21) Appl. No.: 14/349,890

(22) PCT Filed: Oct. 5, 2012
(86) PCT No.: PCT/EP2012/069762

§ 371 Date: Apr. 4, 2014

(30) Foreign Application Priority Data
Oct. 5, 2011 (EP) 11184038.5

(51) Int. Cl. C07H 15/203

(52) U.S. Cl. CPC C07H 15/203
USPC 536/18.5; 536/4.1

(57) ABSTRACT

Carbohydrates are biomolecules that are involved in a range of biological processes and play key roles in, for instance, host immune response and cellular adhesion. Accordingly, functionalisation of medical devices such as stents, valves, catheters, prostheses and other devices for in vivoimplantation with carbohydrates is an area in which considerable interest is developing. Disclosed herein are surfaces having carbohydrates immobilised thereon. The carbohydrate has a linker moiety covalently bound thereto and the linker moiety has a carbon atom that forms a covalent bond with an atom on the target surface. The carbon based bond is a strong, non-hydrolysable covalent bond. Diazonium salts are utilised to produce the functionalised surfaces and they are particularly advantageous as they result in non-toxic readily escapable by-products

### FIELD OF THE INVENTION

The present invention relates to carbohydrates, and in particular carbohydrate coated surfaces which have a myriad of potential uses, inter alia improving biocompatibility of medical devices. Disclosed herein are surface bound carbohydrates showing different bonding to those of the prior art and methods for achieving same.

### BACKGROUND TO THE INVENTION

Carbohydrates are biomolecules that are involved in a range of biological processes and play key roles in, for instance, host immune response and cellular adhesion. Surface functionalisation of carbon-coated devices with carbohydrates can modulate/improve biocompatibility, cell adhesion and/or immune response, thus allowing control over a variety of processes, such as inflammation, resistance to infection and bio-film formation.

Accordingly, functionalisation of medical devices such as stents, valves, catheters, prostheses and other devices for in vivo implantation with carbohydrates is an area in which considerable interest is developing.

International Patent Application Publication No. WO 2010/066049 discloses a methodology for carbohydrate functionalisation of stainless steel surfaces for use in implants and biodevices. The stainless steel must be first coated with a layer of silica as a platform for functionalisation. Carbohydrates containing trialkoxysilane derivatives as chemical handles for surface grafting were prepared and attached to the surface via silanol condensation. This methodology is problematic because it necessitates the use of silica, which is not an ideal coating material for biodevices, and the resulting alkoxy silane bonds are hydrolysable.

International Patent Application Publication No. WO 2008/003298, describes functionalised coatings linked to a stent. The procedure is indicated as being suitable for metals, ceramics and, especially, polymers. The method disclosed involves silanization of the stent surface followed by carbodiimide coupling to an oligosaccharide (e.g. desulfated reacetylated heparin). Again, the technology involves silane coatings and it requires the use of coupling agents such as carbodiimide resulting in hydrolysable bonds.

Functionalisation of carbon nanomaterials such as carbon nanotubes, with carbohydrates has been described and a number of methodologies have been employed for their functionalisation. B. K. Gorityala, J. M. Ma, X. Wang, P. Chen and X. W. Liu, “Carbohydrate functionalized carbon nanotubes and their applications”, Chem. Soc. Rev., 2010, 39, 2925-2934 summarises the advances that have been made in this field. Most functionalisation strategies for carbon nanomaterials involve reactions with carboxylic acid residues on the surface via coupling agents (e.g. carbodiimides). These carboxylic acid groups are introduced on the surface by exposing the carbon nanomaterials to harsh oxidising conditions. However, such an approach has the attendant disadvantage that harsh oxidising conditions are unsuitable for carbon coated biodevices and may result in etching and degradation of the device. Moreover, and as discussed above, tethering of the carbohydrate to the carbon surface requires the use of synthetic coupling agents.

The prior art invariably discloses carbohydrates bonded to surfaces by means of hydrolysable alkoxy silanes, ester linkages or amide linkages. Moreover, the formation of ester linkages and amide linkages necessitates the use of activating, synthetic coupling agents which add additional synthetic complexity to the process of immobilising carbohydrates to a surface in addition to generating by-products, which have to be removed from the end product.

The present invention aims to address these shortcomings by providing a method of immobilising a carbohydrate on a surface through strong, non-hydrolysable covalent bonds, whilst producing non-toxic readily escapable by-products.

### SUMMARY OF THE INVENTION

The present invention allows for the immobilisation or attachment of carbohydrates to surfaces via covalent bonds, which are robust and resistant to hydrolysis. Moreover, the present invention provides a process for achieving same. In particular, the process operates at a low cost, it is mild and it produces non-toxic by-products.

Accordingly, in a first aspect the present invention provides for a method of immobilising a carbohydrate on a surface, the method comprising:
i) providing a carbohydrate having a linker moiety covalently bound thereto, wherein the linker moiety comprises carbon atom bonded to a diazonium cation; and
ii) reacting the diazonium cation with the surface, such that reduction of the diazonium cation results in the carbon atom of the linker moiety forming a covalent bond with an atom on the surface.

Advantageously, reduction of the diazonium salt liberates gaseous nitrogen as a by-product. This is particularly beneficial because gaseous nitrogen is non-toxic and does not have to be manually separated from the end products, thus, greatly simplifying the process.

As used herein the term carbohydrate shall be construed as relating to natural and synthetic monosaccharides, disaccharides, oligosaccharides and polysaccharides. It will be understood that the term carbohydrate includes molecules that have a 5 and 6 membered saturated ring having the substituents on the ring such as the groups as defined herein and the ring contains one heteroatom, which forms part of the ring structure. Suitably, the heteroatom may be O, S or N. Accordingly, thiosugars and iminosugars also fall within the scope of the invention. A natural carbohydrate shall be considered as an organic compound (synthesised de novo or isolated from a natural source), which is capable of being biosynthesised by living organisms, and which has the empirical formula Cₘ(H₂O)ₙ where m and n can be the same or different. Notable exceptions falling outside this empirical formula, but embraced by the present invention nonetheless, are sialic acids and deoxy carbohydrates such as rhamnose, fucose, deoxy ribose, and larger complex carbohydrates comprising either sialic acids or deoxy carbohydrates. The skilled person will appreciate that oligonucleotides, nucleic acids or glycoproteins are not encompassed by the meaning of carbohydrate as intended herein.

The term synthetic carbohydrate is defined as including chemically modified natural carbohydrates and chemically modified non-natural carbohydrates, as defined above, synthesised de novo. A suitable, non-limiting example of synthetic carbohydrates embraced by the present invention is illustrated by the general formula (I) below:

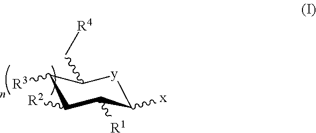

wherein n can be 0 or 1;
X can be OH, SH, NH₂ or CH₃ or when X is part of a fused ring system, X can be O, S, NH, CH, or CH₂;
Y can be O, N, or S; and
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate; or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C. Y is O is preferred.

A further example is illustrated by the general formula (I*) below:

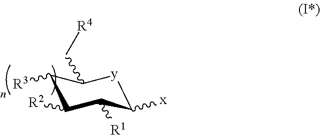

wherein n can be 0 or 1;
X can be OH, SH, NH₂ or CH₃ or when X is part of a fused ring system, X can be O, S, NH, CH or CH₂;
Y can be O, N, or S; and
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate; with the proviso that at least one of R¹, R², R³, and R⁴ is —OH;
or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C.

Suitably, when Y is N or S, it is preferred that at least two of R¹, R², R³, and R⁴ are —OH. More preferably still, at least three of R¹, R², R³, and R⁴ are —OH. More preferably still, all four of R¹, R², R³, and R⁴ are —OH.

As used herein, a diazonium cation bonded to a carbon atom refers to a compound of the formula:

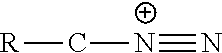

wherein R represents the remainder of the linker moiety. The diazonium cation may have an associated anion. Suitable anions include organic anions, and inorganic anions. For example, halide anions or BF₄—.

With reference to the method of the present invention, the carbon atom in the linker moiety may be a component of an aromatic or aryl ring and the diazonium cation may be an aryl diazonium cation. As will be understood by the skilled person when reading that the carbon atom in the linker moiety may be a component of an aryl ring, it will be understood that the carbon atom forms part of the aryl ring itself. For example, suitable aryl diazonium cations include phenyl diazonium cations of the formula:

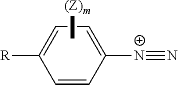

wherein R represents the remainder of the linker moiety
m can be 0 to 4; and
each Z can be independently selected from hydroxy, Cl, Br, I, F, cyano, C₁-C₅ alkoxy, and C₁-C₅ thioalkoxy.

The surface of the present invention will preferably be a reducing surface relative to the diazonium cation, i.e. the surface will provide electrons to reduce the diazonium cation. For example, electron donation from a surface state to the molecular state, i.e. the diazonium cation, should be thermodynamically favoured.

For surfaces where the standard reduction potential is measurable, the diazonium cation utilised in the method of the present invention should have a standard reduction potential greater than the particular surface to which the carbohydrate is to be bound. References to standard reduction potentials in this specification indicate the tendency of a species to acquire electrons and thereby be reduced. Standard reduction potentials are measured under standard conditions: 25° C., 1 M concentration, a pressure of 1.01325×10⁵ Pa (1 atm) and elements in their pure state.

Alternatively, the reduction of the diazonium cation can be driven electrochemically. Where the reduction of the diazonium cation is driven electrochemically the surface should be conducting. In this case the surface will be selected from the group consisting of metals and semiconductors.

Suitable surfaces for use in the method of the present invention may be selected from the group consisting of diamond like carbon, amorphous carbon, hydrogenated tetrahedral carbon, glassy carbon, vitreous carbon, turbostratic carbon; carbon blacks; single crystal diamond, nanocrystalline diamond, polycrystalline diamond, doped or undoped graphene, doped or undoped polycrystalline graphite, doped or undoped highly ordered graphite, doped or undoped graphite oxide, doped or undoped carbon nanotubes, doped or undoped silicon carbide, doped or undoped titanium carbide, metals including Ti, V, Cr, Mn, Fe, Co, Ni, Cu, Zn, Zr, Nb, Mo, Tc, Ru, Rh, Pd, Ag, Cd, Ta, W, Ir, Pt, Au, Hg, In, Sn, Pb, Al, Bi, Tl, Ga, Si or Ge and alloys thereof containing at least one of said metals, such as stainless steels, brasses, bronzes or nickel alloys (e.g. nitinol, hastelloys); GaAs, ITO (indium tin oxide), tin oxide, SiO₂, titanium oxide, iron oxides, manganese oxides, zinc oxides, polymers or plastics such as, polystyrene, polythene, nylon, polytetrafluoroethylene (PTFE), polyestersulfone, polyethyleneterephthalate (PET), polyethersulfone (PES), polyvinyl chlorides (PVC), polystyrenes (PS), polyesters, polyepoxides, polyacetates (e.g. polyvinylacetate), polyethylene oxide, polymethylene oxide, polyphenyl oxide, silicones, polybutadiene, polyacrilonitrile, polypropylene (PP), polyethylene (PE), polyvinylidenefluoride (PVDF), polybutylene (PB), Perfluoroalkoxy (PFA), polytetrafluoroethylene (PTFE), fluorinated ethylene propylene (FEP), ethylene chlorotrifluoroethylene (ECTFE), ethylene trifluoroethylene (ETFE), polycarbonates (PC), polyestersulfone (PES), polysulfones (e.g. polyethersulfone), Polyetheretherketone (PEEK), Polyetherimide (PEI), polyamides (e.g. Nylon, Aramids), polyimides (e.g. Vespel), poly(vinyl alcohol) (PVA), polyacrylics (e.g. PMMA, PAA), polyoxymethylenes (POM), polyurethanes, polyethylene terephthalate (PET), polytrimethylene terephthalate (PTT), polybutyrate, melamine and combinations thereof.

Particularly preferred metal and alloys are Ni, Cu, Fe, Au, Ag, Ti, Zn, Pd, Co, Pt, and alloys thereof containing at least one of said metals, such as stainless steel; Si, Ge, GaAs, ITO (indium tin oxide), tin oxide, SiO₂, titanium oxide, iron oxides, manganese oxides, zinc oxides, polystyrene, polythene, nylon, polytetrafluoroethylene (PTFE), polyestersulfone, polyethyleneterephthalate (PET), and combinations thereof.

It will be appreciated that the nature of the atom on the surface to which the carbon of the linker moiety is covalently bound, depends on the surface itself. For example, in carbon based surfaces this atom is C, whereby a C—C covalent bond is formed. For metal or metal oxide surfaces this atom is typically an O, whereby a C—O covalent bond is formed.

In a preferred embodiment, the surface comprises carbon such that the linker moiety is bonded to the surface by means of a C—C bond. Advantageously, this results in particularly strong bonding of the carbohydrate to the surface by means of a C—C bond, which is resistant to hydrolysis.

Moreover, the increasing popularity and use of carbon materials as biocompatible surfaces on implantable substrates such as medical- and biodevices, for example stents, valves, catheters, and prostheses makes the method of the present invention particularly attractive on account of its compatibility with and ease of applicability to carbon surfaces.

Suitable carbon based surfaces may be selected from diamond like carbon, amorphous carbon, hydrogenated tetrahedral carbon, glassy carbon, vitreous carbon, turbostratic carbon; carbon blacks; single crystal diamond, nanocrystalline diamond, polycrystalline diamond, doped or undoped graphene, doped or undoped polycrystalline graphite, doped or undoped highly ordered graphite, doped or undoped graphite oxide, doped or undoped carbon nanotubes, doped or undoped silicon carbide, doped or undoped titanium carbide, and combinations thereof.

With further reference to the method of the present invention, the linker moiety may be covalently bound to the carbohydrate by means of a glycosidic bond. The glycosidic bond may be selected from the group consisting of an O-glycosidic bond, an S-glycosidic bond, an N-glycosidic bond and a C-glycosidic bond. The glycosidic bond may be an anomeric glycosidic bond.

The linker moiety may be selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₃-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy.

In one embodiment, the carbohydrate having a linker moiety covalently bound thereto, wherein the linker moiety comprises carbon atom bonded to a diazonium cation, may be of the following general formula:

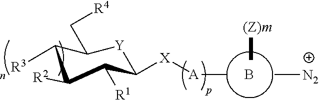

wherein n can be 0 or 1;
X can be O, S, NH or CH₂;
Y can be O, N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate; or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;
B can be C₅-C₂₀ aryl, or C₃-C₂₀ heteroaryl;
A can be C₁-C₂₀ aliphatic, or C₁-C₂₀ heteroaliphatic; and
p can be 0 or 1. Y is O is preferred.

A further example is illustrated by the following general formula:

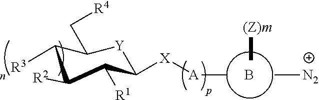

wherein n can be 0 or 1;
X can be O, S, NH or CH₂;
Y can be N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate, with the proviso that at least one of R¹, R², R³, and R⁴ is —OH;
or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;
B can be C₅-C₂₀ aryl, or C₃-C₂₀ heteroaryl;
A can be C₁-C₂₀ aliphatic, or C₁-C₂₀ heteroaliphatic; and
p can be 0 or 1.

Suitably, when Y is N or S, it is preferred that at least two of R¹, R², R³, and R⁴ are —OH. More preferably still, at least three of R¹, R², R³, and R⁴ are —OH. More preferably still, all four of R¹, R², R³, and R⁴ are —OH. In one embodiment, it is preferred that R⁴ is —OH.

B can be can be selected from the group consisting of phenyl, pyridyl, thienyl, pyrollyl, pyrazyl, pyrimidyl, imidazolyl, indolyl, quinolyl, and isoquinolyl. For example, B may be phenyl.

With reference to the method of the present invention the step of reacting the diazonium cation with the surface may comprise dip coating or immersing the surface in a solution of, or a suspension of the diazonium cation in a solvent. Suitable solvents may be selected from the group consisting of water, acetonitrile, C₁-C₂₀ alcohols, tetrahydrofuran, C₁-C₂₀ formamides, C₁-C₂₀ chlorinated hydrocarbons, and ionic liquids. In a further embodiment, the solvent may further comprise a reducing agent suspended or dissolved therein for increasing the rate of reduction of the diazonium cation.

With reference to the method of the present invention, the step of reacting the diazonium cation with the surface may further comprise the steps of:

subjecting the diazonium cation and surface to ultrasonication, and/or

subjecting the diazonium cation and surface to microwave heating. Advantageously, such steps may increase the rate at which the carbon atom of the linker moiety forms a covalent bond with an atom on the surface.

The invention further provides for a surface with a carbohydrate immobilised thereon obtainable by the method of the present invention.

In a further aspect, the present invention provides for a surface having a carbohydrate immobilised thereon,

the carbohydrate having a linker moiety covalently bound thereto, the linker moiety disposed between the surface and the carbohydrate, and

the linker moiety comprising a carbon atom that forms a covalent bond with an atom on the surface, wherein

the carbon atom of the linker moiety that forms a covalent bond with an atom on the surface is not substituted with a ═O moiety.

That is, the carbon atom of the linker that forms a covalent bond with an atom on the surface is not a carbonyl carbon and it is not a component of an ester moiety or an amide moiety.

In a further embodiment, the carbon atom of the linker moiety that forms a covalent bond with an atom on the surface is not substituted with a ═O, ═S or a ═N moiety, i.e. the carbon atom of the linker that forms a covalent bond with an atom on the surface is not a carbonyl carbon, a thiocarbonyl carbon, nor an imino carbon and it is not a component of an ester moiety, thioester moiety, amide moiety, or an amidine moiety.

Advantageously, the present invention provides for a surface wherein the carbohydrate is not immobilised thereon by means of an alkoxysilane, an ester linkage or an amide linkage. Such linkages are susceptible to hydrolysis and the present invention provides for more robust bonding of carbohydrates to the surface.

In particular, the carbon atom of the linker moiety that forms a covalent bond with an atom on the surface may be a component of an aromatic ring.

Suitable surfaces may be selected from the group consisting of diamond like carbon, amorphous carbon, hydrogenated tetrahedral carbon, glassy carbon, vitreous carbon, turbostratic carbon; carbon blacks; single crystal diamond, nanocrystalline diamond, polycrystalline diamond, doped or undoped graphene, doped or undoped polycrystalline graphite, doped or undoped highly ordered graphite, doped or undoped graphite oxide, doped or undoped carbon nanotubes, doped or undoped silicon carbide, doped or undoped titanium carbide, metals including Ti, V, Cr, Mn, Fe, Co, Ni, Cu, Zn, Zr, Nb, Mo, Tc, Ru, Rh, Pd, Ag, Cd, Ta, W, Ir, Pt, Au, Hg, In, Sn, Pb, Al, Bi, Tl, Ga, Si or Ge and alloys thereof containing at least one of said metals, such as stainless steels, brasses, bronzes or nickel alloys (e.g. nitinol, hastelloys); GaAs, ITO (indium tin oxide), tin oxide, SiO₂, titanium oxide, iron oxides, manganese oxides, zinc oxides; polymers or plastics such as, polystyrene, polythene, nylon, polytetrafluoroethylene (PTFE), polyestersulfone, polyethyleneterephthalate (PET), polyethersulfone (PES), polyvinyl chlorides (PVC), polystyrenes (PS), polyesters, polyepoxides, polyacetates (e.g. polyvinylacetate), polyethylene oxide, polymethylene oxide, polyphenyl oxide, silicones, polybutadiene, polyacrilonitrile, polypropylene (PP), polyethylene (PE), polyvinylidenefluoride (PVDF), polybutylene (PB), Perfluoroalkoxy (PFA), polytetrafluoroethylene (PTFE), fluorinated ethylene propylene (FEP), ethylene chlorotrifluoroethylene (ECTFE), ethylene trifluoroethylene (ETFE), polycarbonates (PC), polyestersulfone (PES), polysulfones (e.g. polyethersulfone), Polyetheretherketone (PEEK), Polyetherimide (PEI), polyamides (e.g. Nylon, Aramids), polyimides (e.g. Vespel), poly(vinyl alcohol) (PVA), polyacrylics (e.g. PMMA, PAA), polyoxymethylenes (POM), polyurethanes, polyethylene terephthalate (PET), polytrimethylene terephthalate (PTT), polybutyrate, melamine and combinations thereof.

Particularly preferred metal and alloys are Ni, Cu, Fe, Au, Ag, Ti, Zn, Pd, Co, Pt, and alloys thereof containing at least one of said metals, such as stainless steel; Si, Ge, GaAs, ITO (indium tin oxide), tin oxide, SiO₂, titanium oxide, iron oxides, manganese oxides, zinc oxides, polystyrene, polythene, nylon, polytetrafluoroethylene (PTFE), polyestersulfone, polyethyleneterephthalate (PET), and combinations thereof.

In a preferred embodiment, the surface comprises carbon such that the linker moiety is bonded to the surface by means of a C—C bond. Advantageously, this results in particularly strong bonding of the carbohydrate to the surface by means of a C—C bond, which is resistant to hydrolysis.

Moreover, the increasing popularity and use of carbon materials as biocompatible surfaces on implantable substrates such as medical- and biodevices, for example stents, valves, catheters, and prostheses makes the method of the present invention particularly attractive on account of its compatibility with and ease of applicability to carbon surfaces.

Suitable carbon based surfaces may be selected from diamond like carbon, amorphous carbon, hydrogenated tetrahedral carbon, glassy carbon, vitreous carbon, turbostratic carbon; carbon blacks; single crystal diamond, nanocrystalline diamond, polycrystalline diamond, doped or undoped graphene, doped or undoped polycrystalline graphite, doped or undoped highly ordered graphite, doped or undoped graphite oxide, doped or undoped carbon nanotubes, doped or undoped silicon carbide, doped or undoped titanium carbide and combinations thereof.

The linker moiety may be covalently bound to the carbohydrate by means of a glycosidic bond. The glycosidic bond may be selected from the group consisting of an O-glycosidic bond, an S-glycosidic bond, an N-glycosidic bond and a C-glycosidic bond. The glycosidic bond may be an anomeric glycosidic bond.

The linker moiety may be selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₃-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy.

The carbohydrate and linker immobilised on the surface of the present invention may be of the general formula:

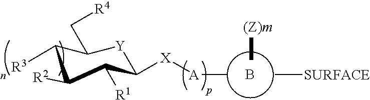

wherein n can be 0 or 1;
X can be O, S, NH or CH₂;
Y can be O, N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate; or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;
B can be C₅-C₂₀ aryl, or C₃-C₂₀ heteroaryl;
A can be C₁-C₂₀ aliphatic, or C₁-C₂₀ heteroaliphatic; and
p can be 0 or 1.

A further example is illustrated by the following general

formula:

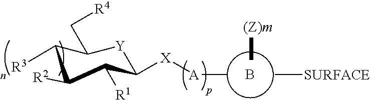

wherein n can be 0 or 1;
X can be O, S, NH or CH₂;
Y can be O, N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate, with the proviso that at least one of R¹, R², R³, and R⁴ is —OH;
or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;

B can be C₅-C₂₀ aryl, or C₃-C₂₀ heteroaryl;
A can be C₁-C₂₀ aliphatic, or C₁-C₂₀ heteroaliphatic; and
p can be 0 or 1.

Suitably, when Y is N or S, it is preferred that at least two of R¹, R², R³, and R⁴ are —OH. More preferably still, at least at least three of R¹, R², R³, and R⁴ are —OH. More preferably still, all four of R¹, R², R³, and R⁴ are —OH. In some embodiment, it is preferred that R⁴ is —OH.

B can be can be selected from the group consisting of phenyl, pyridyl, thienyl, pyrollyl, pyrazyl, pyrimidyl, imidazolyl, indolyl, quinolyl, and isoquinolyl. For example, B may be phenyl.

In a further aspect, there is provided for a method of immobilising a carbohydrate to a surface, the method comprising:
i) providing a carbohydrate having a linker moiety covalently bound thereto, wherein the linker moiety comprises carbon atom bonded to a diazonium cation, wherein the carbon atom in the linker moiety that is bonded to the diazonium cation is a component of an aromatic or aryl ring and the diazonium cation is an aryl diazonium cation; and
ii) reacting the diazonium cation with the surface, such that reduction of the diazonium cation results in the carbon atom of the linker moiety forming a covalent bond with an atom on the surface.

In yet a further aspect, the present invention provides for use of a molecule of the general formula (II) in a method of immobilising a carbohydrate on a surface:

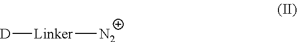

wherein D is a carbohydrate moiety; and
the linker moiety may be selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₃-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy,
wherein the diazonium cation is bonded to a carbon atom in the linker moiety.

Preferably, the carbon atom in the linker moiety that is bonded to the diazonium cation is a component of an aromatic or aryl ring and the diazonium cation is an aryl diazonium cation.

Preferably, the carbohydrate is selected from the group consisting of: natural and synthetic monosaccharides, disaccharides, oligosaccharides and polysaccharides,

In one embodiment, the carbon atom in the linker moiety is a component of an aryl ring and the diazonium cation is an aryl diazonium cation.

The molecule may be of the general formula (IIa):

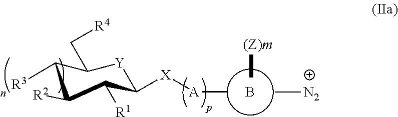

wherein n can be 0 or 1;
X can be O, S, NH or CH₂;
Y can be O, N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate; or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;
B can be C₅-C₂₀ aryl, or C₃-C₂₀ heteroaryl;
A can be C₁-C₂₀ aliphatic, or C₁-C₂₀ heteroaliphatic; and
p can be 0 or 1.

Another example is illustrated by the general formula (IIa*):

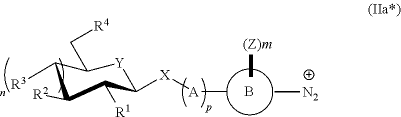

wherein n can be 0 or 1;
X can be O, S, NH or CH₂;
Y can be O, N, or S;
each of R¹, R², R³, and R⁴ can be the same or different and may be selected from the group consisting of OH, H, NH₂, F, SH, C₂-C₃₀ carbon esters, C₂-C₃₀ amides, C₂-C₃₀ phosphate esters, C₁-C₁₀ amino alkyl, C₂-C₂₀ bisalkyl amino, C₁-C₁₀ alkoxy, C₁-C₁₀ thioalkoxy, C₅-C₂₀ aryloxy, C₅-C₂₀ thioaryloxy, C₅-C₂₀ heteroaryloxy, C₅-C₂₀ thioheteroaryloxy, an O-glycosidic linkage to another natural or synthetic carbohydrate, an S-glycosidic linkage to another natural or synthetic carbohydrate, an N-glycosidic linkage to another natural or synthetic carbohydrate or a C-glycosidic linkage to another natural or synthetic carbohydrate, with the proviso that at least one of R¹, R², R³, and R⁴ is —OH; or
each of R¹, R², R³ and R⁴ and the carbon atoms to which they are attached may independently define a C₃-C₂₀ fused heterocycle along with X, when X comprises one of O, N, or S, or a C₃-C₂₀ fused carbocycle along with X, when X comprises C;
m can be 0 to 4;
each Z can be independently selected from hydroxy, Cl, Br, I, F, cyano, C₁-C₅ alkoxy, and C₁-C₅ thioalkoxy;
B can be C₅-C₂₀ aryl, or C₃-C₂₀ heteroaryl;
A can be C₁-C₂₀ aliphatic, or C₁-C₂₀ heteroaliphatic; and
p can be 0 or 1.

Suitably, when Y is N or S, it is preferred that at least two of R¹, R², R³, and R⁴ are —OH. More preferably still, at least at least three of R¹, R², R³, and R⁴ are —OH. More preferably still, all four of R¹, R², R³, and R⁴ are —OH. In some embodiment, it is preferred that R⁴ is —OH.

B can be can be selected from the group consisting of phenyl, pyridyl, thienyl, pyrollyl, pyrazyl, pyrimidyl, imidazolyl, indolyl, quinolyl, and isoquinolyl. For example, B may be phenyl.

Advantageously, the carbohydrate diazonium material can be isolated (and stored for subsequent use at a later stage) or directly carried through to the next stage of the reaction process without purification, i.e. reaction with the surface such that the carbon atom of the linker bonded to the diazonium cation now becomes bonded to the surface.

Surfaces of the present invention may find a myriad of potential uses. Suitable applications are disclosed below:

Biocompatible coatings—the surfaces may be utilised to improve the tolerance and immunological response to matter implanted in vivo such as implants and devices. For instance, carbohydrate coatings for stents, heart valves, catheters, electrodes, prostheses, dental implants, biomechanical parts (e.g. screws), ophthalmic devices (e.g. lenses), orthodontic and orthopaedic implants or any other artificial device for implantation into a living organism. The technology may also be applied to improve tolerance to sub-cutaneous pigments (tattoo inks based on carbons or oxides);

Antirestenotic coatings, for example for catheters and stents;

Bioactive coatings that actively enhance, improve or modulate biological response via carbohydrate-directed interactions;

Anti-biofouling coatings that prevent or minimize adhesion, attachment, build-up or proliferation of cells, microorganisms or biofilms in any application where surfaces are susceptible to biological fouling. Possible applications are to minimize infection associated with catheters, needles, guidewires, bulking agents, orthodontic and orthopaedic implants;

Prolonging the lifetime of materials _{[AG1]}such as sensors, tools, and pipes that come into contact with natural waters or soils (e.g. antifouling of aquatic sensors, agricultural tools and utensils, veterinary equipment);

Antimicrobial and antifungal coatings that prevent the attachment of bacterial and fungus, for example in medical sterilisation, food and beverage manufacturing, processing, dispensing, preservation and packaging, textiles and utensils for agriculture and veterinary applications;

Surfaces and scaffolds for tissue culture and engineering—carbohydrate coatings on various surfaces can be used to modulate tissue and cell growth and attachment and can therefore be applied to tissue engineering;

Surface patterning of bioactive entities—the carbohydrates are first patterned via any suitable method, for example solution methods or microcontact printing, and may be subsequently used to direct the adhesion/physisorption of biomolecules such as enzymes, proteins, antibodies; or of cells such as bacteria or viruses;

Screening Processes—carbohydrate immobilization on screening platforms such as screening biochips or arrays can be used as a platform technology for investigating, detecting, diagnosing or screening of glycoprotein interactions. This finds applications in antibody screening, vaccine research, small drug screening, cell surface profiling of expressed carbohydrates or lectins. Carbohydrate arrays can be used for the screening of synthetic carbohydrates as therapeutics;

Drug and nanoparticle delivery—carbohydrate coatings can be used for targeted delivery of either small molecules with therapeutic properties or of nanoparticles with therapeutic/diagnostic properties. Possible applications of this technology include carbohydrate coating of particles for aerosol delivery, particles as contrast agents, nanodiamonds and nanotubes or polymer capsules for therapeutic and diagnostic applications;

Biochips for rapid diagnostics—carbohydrate arrays as described above can be used for screening of disease markers, for instance for early-stage detection of disease;

Immobilization of enzymes and cells for catalysis and bioreactor applications; and

Separation processes—for instance in the modification of membranes or stationary phases for chemical separation and extraction processes.

The present invention further provides for a device for in-vivo implantation comprising a surface according to the present invention.

The present invention also provides for a diagnostic kit comprising a surface according to the present invention.

In yet a further aspect, the present invention provides for use of a surface according to the present invention in the manufacture of at least one of:

a material for in vivo implantation;

a bio-fouling resistant material;

a scaffold for tissue culture and engineering;

a screening array for monitoring glycoprotein interactions;

a diagnostic kit;

a drug delivery vehicle;

a chromatographic stationary phase;

equipment/materials involved in food manufacture, processing and/or dispensing;

a sensor such as an aquatic, bioanalytical and/or electrochemical;

surgical/veterinary utensils; or

a tool for agriculture and/or livestock.

In yet a further aspect still the present invention provides for use of a surface according to the present invention in at least one application selected from in vivo implantation; bio-fouling resistant materials; tissue scaffolds; diagnostics; drug delivery; antifouling coatings; glycoarrays; filtration membranes; biomedical devices such as implants, sensors, catheters, guidewires, and/or dental parts; equipment/materials involved in food manufacture, processing and/or dispensing; sensors such as aquatic, bioanalytical, and/or electrochemical; solid phases for separation/filtration; surgical/veterinary utensils; and tools for agriculture and/or livestock.

As used herein, the term Cₓ-C_{y} aliphatic refers to linear, branched, saturated and unsaturated hydrocarbon chains comprising Cₓ-C_{y} carbon atoms (and includes Cₓ-C_{y} alkyl, Cₓ-C_{y} alkenyl and Cₓ-C_{y} alkynyl). Cₓ-C_{y} heteroaliphatic refers to linear, branched, saturated and unsaturated hydrocarbon chains comprising Cₓ-C_{y} carbon atoms, wherein the carbon atoms are interspaced with heteroatoms such as O, N, and S in no regular order or sequence. Cₓ-C_{y} heteroaliphatic shall be construed as covering polyethers and polythioethers.

Similarly, references to Cₓ-C_{y} alkyl, Cₓ-C_{y} alkenyl and Cₓ-C_{y} alkynyl include linear and branched Cₓ-C_{y} alkyl, Cₓ-C_{y} alkenyl and Cₓ-C_{y} alkynyl.

As used herein, the term “Cₓ-C_{y} cycloaliphatic” refers to unfused, fused, spirocyclic, polycyclic, saturated and unsaturated hydrocarbon rings comprising Cₓ-C_{y} carbon atoms (and includes Cₓ-C_{y} cycloalkyl, Cₓ-C_{y} cycloalkenyl and Cₓ-C_{y} cycloalkynyl).

As used herein, the term aryl/aromatic refers to an aromatic carbocyclic structure which is monocyclic or polycyclic and is unfused or fused. As used herein, the term heterocycle refers to cyclic compounds having as ring members, atoms of at least two different elements. The cyclic compounds may be monocyclic or polycyclic, and unfused or fused.

As used herein, the term heteroaromatic/heteroaryl refers to an aromatic heterocyclic structure having as ring members, atoms of at least two different elements. The heterocycle may be monocyclic or polycyclic, and unfused or fused.

The compounds of the present invention may be found or isolated in the form of esters, salts, hydrates or solvates—all of which are embraced by the present invention.

Where suitable, it will be appreciated that all optional and/or preferred features of one embodiment of the invention may be combined with optional and/or preferred features of another/other embodiment(s) of the invention.

### BRIEF DESCRIPTION OF THE DRAWINGS

Additional features and advantages of the present invention are described in, and will be apparent from, the detailed description of the invention and from the drawings in which:

FIG. 1 illustrates infrared reflectance absorption spectra (IRRAS) of the bonded carbohydrate on copper. Absorption peaks in the surface spectrum match those of the precursor molecule indicating that rhamnose is successfully tethered and available at the interface.

FIG. 2 illustrates exemplary glycosyl amino compounds of the invention.

FIG. 3 illustrates a Peg-Triazole linked compound of the invention.

FIG. 4a to f illustrates IIRAS spectra on various surfaces (amorphous carbon, hydrogenatied amorphous carbon, Cu, Ti, Au, and brass; FIG. 4g shows: ATR of Galactose precursor covalently bound at amorphous carbon surface before (bottom) and after (top) deacetylation.

FIG. 5 illustrates ATR of the starting material: Galactose-Amine.

FIG. 6a-c illustrates characterisation data for the galactose series of compounds.

FIG. 7a & b illustrates characterisation data for the rhamnose series of compounds.

FIG. 8a-c illustrates characterisation data for the mannose series of compounds.

FIG. 9 illustrates characterisation data for the glucose series of compounds.

### DETAILED DESCRIPTION OF THE DRAWINGS

It should be readily apparent to one of ordinary skill in the art that the examples disclosed herein below represent generalised examples only, and that other arrangements and methods capable of reproducing the invention are possible and are embraced by the present invention.

A general schematic for the synthesis of the amino precursors to the diazonium substituted carbohydrates in given in Scheme 1 below.

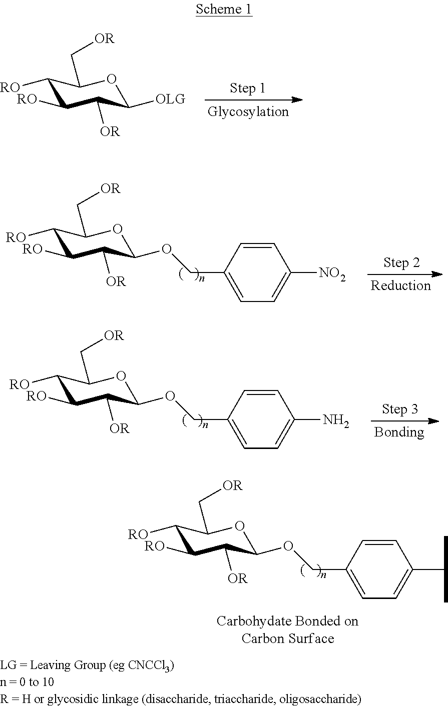

In particular, the anomeric position on the saccharide is substituted with a leaving group. The leaving group is eliminated and it replaced by an aromatic nitro compound, wherein the phenyl ring is substituted with an aliphatic chain terminating with oxygen. The nitro functional group is subsequently reduced to the corresponding amine, whereupon it is subjected to a diazotisation procedure.

The resulting carbohydrate diazonium material can be isolated (and stored for subsequent use at a later stage) or carried on to the next stage of the reaction process, i.e. reaction with the surface such that the carbon atom of the linker bonded to the diazonium cation now becomes bonded to the surface.

A schematic for the synthesis of particular amino precursors to the diazonium substituted carbohydrates in given in Schemes A and B below.

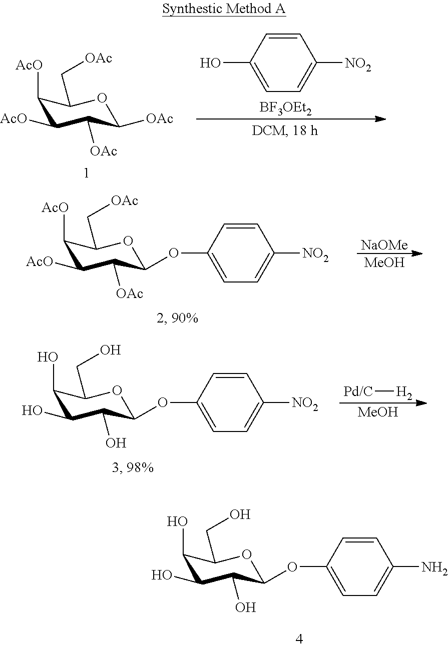

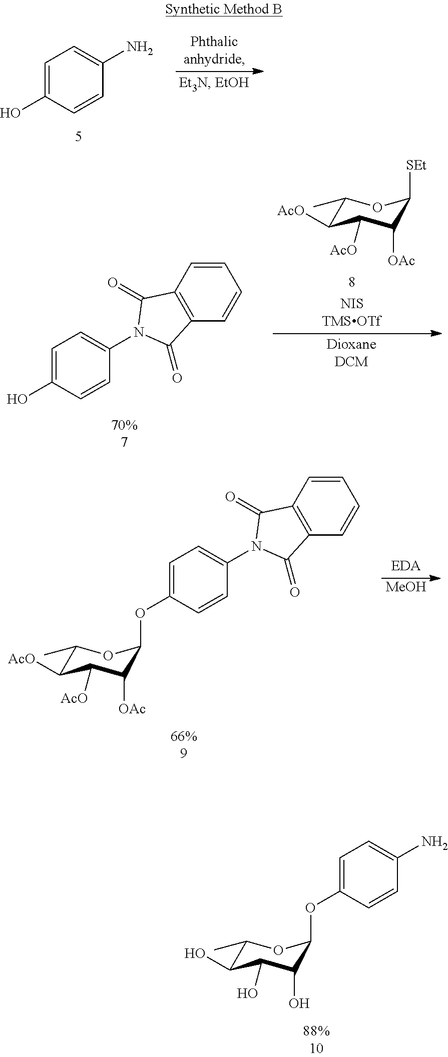

### Synthetic Method A

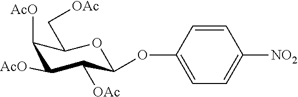

### 2, 3, 4, 6-tetra-O-acetyl-1-(4-nitrophenoxy)-β-D-galactopyranoside (2)

1, 2, 3, 4, 6-penta-O-acetyl-β-D-galactose (1.0 g, 2.56 mmol) and 4-nitrophenol (379 mg, 2.72 mmol) were dissolved in anhydrous DCM and BF₃.OEt₂ (1.28 mL, 10.5 mmol) added. After overnight reaction the mixture was quenched with 200 mL of sat. aq NaHCO₃, dried with MgSO₄, filtered and concentrated. Purification by column chromatography (hexane:EtOAc 60:40) gave the product as a yellow oil in 90% total yield (1.082 g), 5% alpha anomer yield (53 mg). Data for beta anomer: mp 96-98° C. ¹H-NMR (400 MHz, CDCl₃): δ 8.25, 7.12 (2H, m, Ar), 5.56 (1H, dd, J_{2,1}=7.8 Hz, J_{2,3}=10.4 Hz, H2), 5.51 (1H, J_{4,3}=3.5 Hz, J_{4,5}=0.7 Hz, H4), 5.20 (1H, d, J_{1,2}=7.8 Hz, H1), 5.17 (1H, dd, J_{3,2}=10.4 Hz, J_{3,4}=3.5 Hz, H3), 4.20 (2H, m, H6), 4.18 (1H, m, H5), 2.22, 2.11, 2.11, 2.06 (3H, s, CH₃). NMR (100 MHz, CDCl₃): δ 170.3, 170.1, 170.1, 169.3 (C═O), 161.2, 143.2 (Cq), 98.6 (01), 71.5 (C5), 70.6 (C3), 68.3 (C2), 66.7 (C4), 61.3 (C6), 20.7, 20.7, 20.6, 20.6 (CH₃). HRMS (EI) calculated for C₂₀H₂₃NO₁₂Na 492.1118. found 492.1118. vₘₐₓ (L) 1745, 1592, 1491, 1338 cm⁻¹.

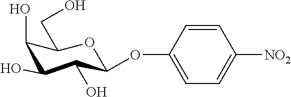

### 1-(4-nitrophenoxy)-β-D-galactopyranoside (3)

2, 3, 4, 6-tetra-O-acetyl-1-(4-nitrophenoxy)-β-D-galactopyranoside (110 mg, 0.23 mmol) was dissolved in MeOH with a catalytic amount of NaOMe. After 18 h the reaction was quenched with a catalytic amount of DOWEX, filtered and concentrated to give the product as an yellow oily solid in 98% yield (69 mg). ¹H-NMR (400 MHz, CDCl₃): δ 8.24, 7.35 (2H, m, Ar), 5.71 (1H, d, J_{1,2}=3.3 Hz, H1), 4.03 (1H, dd, J_{2,1}=3.3 Hz, J_{2,3}=9.8 Hz, H2), 4.01 (1H, m, H4), 3.99 (1H, m, H3), 3.85 (1H, app t, J=5.9 Hz, H5), 3.71 (2H, d, J_{6,5}=6.0 Hz, H6). HRMS (EI) calculated for C₁₂H₁₅NO₈Na 324.0695. found 324.0709.

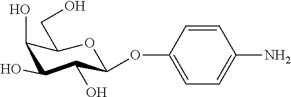

### 1-(4-aminophenoxy)-β-D-galactopyranoside (4)

1-(4-nitrophenoxy)-β-D-galactopyranoside (assumed 70 mg, 0.23 mmol) was dissolved in 1:1 MeOH:THF, a catalytic amount of Pd/C was added and the flask evacuated using a water pump, H₂ was introduced and the stirred reaction was allowed to proceed under H₂ for 48 h then the H₂ was pumped out and air reintroduced. The Pd/C was filtered off and concentrated, NMR analysis revealed that full reduction to the desired amine had not occurred. The product was observed by NMR and mass spec analysis.

### Synthetic Method B

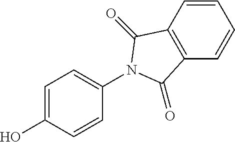 [SMILES: O=C1c2ccccc2C(=O)N1c1ccc(O)cc1]

### N-(4-hydroxyphenyl)phthalimide (7)

4-Aminophenol (1.0 g, 9.18 mmol) and TEA (0.5 mL) were added to a solution of phthalic anhydride (1.5 g, 10.13 mmol) in EtOH (50 mL) and allowed to react at room temperature for 18 h. The mixture was concentrated and the resulting oil resuspended in distilled water and refluxed for 30 minutes and concentrated. The crude product was dissolved in EtOAc, and washed with 0.1N HCl (15 mL) and H₂O (15 mL), dried with MgSO₄ and concentrated to a white powder in 70% yield (3.05 g). mp: 293-295° C. Lit. 291-293° C. ¹H-NMR (400 MHz, DMSO): δ 9.80 (1H, s, OH), 7.92 (4H, m, Ar), 7.21 (2H, d, J=8.7 Hz, Ar), 6.87 (2H, d. J=8.6 Hz, Ar). vₘₐₓ (L) 3405, 1706, 1515, 1117 cm⁻¹.

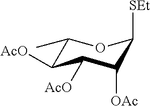

### Ethyl-2,3,4-O-triacetyl-1-thio-L-rhamnopyranoside (8)

1, 2, 3, 4-tetra-O-acetyl-L-rhamnopyranoside (4.0 g, 12.03 mmol) was dissolved in anhydrous DCE (15 mL) at 0° C. under N₂. Ethane thiol (834 μL, 16 mmol) was added followed by BF₃.OEt₂ (7.2 mL, 61 mmol) in increments. After 18 h the mixture was quenched by treatment with sat. NaHCO₃ solution (ca. 20 mL) and solid NaHCO₃. The organic layer was concentrated and purification by column chromatography (hexane:EtOAc 80:20) gave the product as a mixture of anomers in 80% yield. The anomers can be separated but can be used as a mixture. Data for a anomer (crystalline solid): mp 130-132° C. Lit. 135° C. ¹H-NMR (400 MHz, CDCl₃): δ 5.36 (1H, dd, J_{2,1}=1.6 Hz, J_{2,3}=3.4 Hz, H2), 5.26 (1H, dd, J_{3,2}=3.4 Hz J_{3,4}=10.1 Hz, H3), 5.22 (1H, d, J=1.5 Hz, H1), 5.12 (1H, app t, H4), 4.26 (1H, dd, J_{5,6}=6.2 Hz, J_{5,4}=9.7 Hz, H5), 2.67 (2H, m, CH₂), 2.13, 2.08, 2.01 (3H, s, CH₃), 1.32 (3H, app t, CH₃), 1.26 (3H, d, J_{6,5}=6.1 Hz, H6). HRMS (EI) m/z calculated for C₁₄H₂₂O₇SNa 357.0984. found 357.0973.

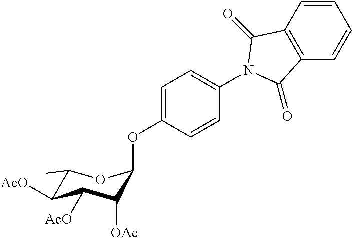

### 1-(4-(1,3-dioxoisoindolin-2-yl)phenoxy)-2,3,4-tri-O-acetyl-α-L-rhamnopyranoside (9)

Ethyl-2,3,4-tri-O-acetyl-1-thio-α-L-rhamnopyranoside (560 mg, 1.68 mmol) was dissolved in 1:1 anhydrous dioxane:DCM (10 mL) under N₂ with 7 (482 mg, 2.02 mmol), NIS (603 mg, 2.68 mmol) and a catalytic amount (ca. 10 μL) of TMS.OTf at 0° C. After 18 h at rt the mixture was quenched with ca. 1 mL of TEA, washed with sat. NaHCO₃ solution (15 mL), dried with MgSO₄ and concentrated. Purification by column chromatography (toluene:acetone 95:5) gave the product as a yellow solid in 66% yield (565 mg). mp 151-152° C. [α_{D}]²⁴ −0.094° (c 0.139, CHCl₃). ¹H-NMR (400 MHz, CDCl₃): δ 7.99, 7.83, 7.40, 7.24 (2H, m, Ar), 5.55 (1H, dd, J_{3,2}=3.5 Hz, J_{3,4}=10.0 Hz, H3), 5.53 (1H, d, J_{1,2}=1.9 Hz, H1), 5.48 (1H, dd, J_{2,1}=1.9 Hz, J_{2,3}=3.5 Hz, H2, 5.20 (1H, app t, H4), 4.02 (1H, m, H5), 2.23, 2.10, 2.07 (3H, s, CH₃), 1.26 (3H, d, J_{6,5}=6.3 Hz, H6). ¹³C-NMR (100 MHz, CDCl₃): δ 170.1, 167.4, 155.4 (Cq), 134.5 (Ar), 131.8, 129.1 (Cq), 128.1 (Ar), 126.2, 125.3 (Cq), 123.8, 116.9 (Ar), 95.7 (01), 70.9 (C4), 69.6 (C2), 68.9 (C3), 67.3 (C5), 20.9, 20.8, 20.8 (CH₃), 17.5 (C6). HRMS (EI) calculated for C₂₆H₂₅NO₁₀Na 534.1376. found 534.1368. vₘₐₓ (L) 1745, 1717, 1609, 1509 cm⁻¹.

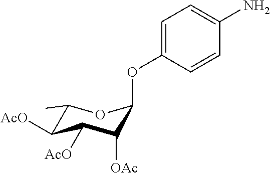

### 1-(4-aminophenoxy)-α-L-rhamnopyranoside (10)

9 (350 mg, 0.69 mmol) was dissolved in MeOH (10 mL) and EDA (1.83 mL, 27.40 mmol) and refluxed for 1 h at 70° C. then concentrated. Column chromatography (hexane:EtOAc:MeOH 40:60:10) gave the product as a pale brown oil in 88% yield (153 mg). [α_{D}]²⁴ −0.136° (c 0.111, MeOH). ¹H-NMR (400 MHz, CDCl₃): δ 6.87, 6.72 (2H, m, Ar), 5.25 (1H, d, J_{1,2}=1.8 Hz, H1), 3.99 (1H, dd, J_{2,1}=1.8 Hz, J_{2,3}=3.5 Hz, H2), 3.84 (1H, dd, J_{3,3}=3.5 Hz, J_{3,4}=9.5 Hz, H3), 3.73 (1H, m, H5), 3.46 (1H, app t, H4), 1.26 (3H, d, J_{6,5}=6.2 Hz, H6). ¹³C-NMR (100 MHz, CDCl₃): δ 149.5, 142.0 (Cq), 117.5, 116.5, (Ar), 99.6 (C1), 72.6 (C4), 70.9 (C3), 70.8 (C2), 69.0 (C5), 16.6 (C6). HRMS (EI) calculated for C₁₂H₁₈NO₅ 256.1185. found 256.1190. vₘₐₓ (L) 3552, 3459, 3372, 1626, 1513 cm⁻¹.

Preparation of the Carbohydrate-Aryldiazonium Derivatives

Carbohydrate-arydiazonium derivatives were prepared from their corresponding amines according to reported literature procedures: D'Amour, M.; Belanger, D., Stability of Substituted Phenyl Groups Electrochemically Grafted at Carbon Electrode Surface Journal of Physical Chemistry B 2003, 107 (20), 4811-4817; and Hermans, A.; Seipel, A. T.; Miller, C. E.; Wightman, R. M., Carbon-Fiber Microelectrodes Modified with 4-Sulfobenzene Have Increased Sensitivity and Selectivity for Catecholamines. Langmuir 2006, 22 (5), 1964-1969.

In short, the amine derivative of the desired compound (X M) was dissolved in tetrafluoroboric acid (2×M). Following cooling to −5° C. a 1×M solution of sodium nitrite in water was added dropwise over 30 min with stirring. If the tetrafluoroborate salt of the carbohydrate-arydiazonium derivatives is to be isolated, the precipitate obtained is filtered by suction, washed with an ice cold ether/methanol mixture (4:1) and cold ethanol, and dried under high vacuum.

Functionalisation of Carbon or Metal Substrates with Carbohydrate-Aryldiazonium Derivatives

(a) from the Isolated Tetrafluoroborate Salt

A solution of the carbohydrate-diazonium tetrafluoroborate of known concentration, generally in the mM range, is made up in a suitable solvent (water if the compounds is water soluble; or, acetonitrile, tetrahydrofuran and alcohols). A clean substrate is immersed in the solution for a noted period of time (typically 5-60 min). Following immersion, the substrate is removed from the reaction vessel, rinsed a number of times in a suitable solvent, sonicated for 30 seconds to remove physisorption products and dried under Argon.

### (b) Through In-Situ Generation of Carbohydrate-Diazonium

Carbohydrate-diazonium cations are generated via in-situ diazotisation of carbohydrate-arylamine derivatives. The diazoniation procedure is carried out as described above (i) however in this protocol the isolation step is replaced with immediate functionalisation of carbon or metal surfaces. These substrates are immersed in the carbohydrate diazonium cation solution for a period of time (typically 5-60 min), removed and washed in a suitable solvent and finally dried under Argon.

FIG. 1 illustrates the infrared reflectance absorption spectra (IRRAS) of the carbohydrate bonded layer on copper. The upper spectrum corresponds to that of the diazonium cation corresponding to rhamnose derivative 10 supra. The lower spectrum illustrates the readout obtained for the same diazonium rhamnose derivative bonded to a copper surface through the aromatic carbon, which in compound 10 is substituted with the amino group. Absorption peaks in the surface spectrum match those of the precursor molecule indicating that rhamnose is successfully tethered to the surface and is available at the interface.

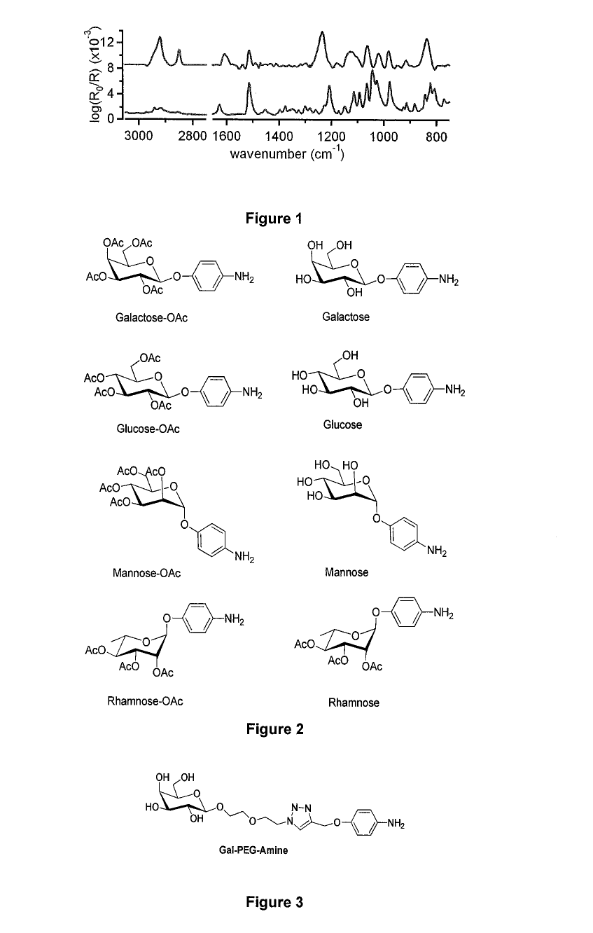

A general strategy for the synthesis of both fully protected (O-acetyl protecting groups) and fully deprotected (all OH's free) glycosyl aromatic amines (precursor molecules for surface functionalisation) has been developed and applied to the synthesis of four protected and four deprotected monosaccharides. Glucose, Galactose, Mannose and Rhamnose have all been prepared in both their protected and deprotetced form. (FIG. 2). The protected sugars are useful in that the ester groups allow for easy detection of the sugar unit on the functionalised surface by FTIR. The general synthetic strategy is outlined in Scheme 2. The key synthetic factor is carrying out the reduction of the nitro group on the protected carbohydrate which allows purification of the amine.

Discussion—Scheme 2 illustrates the synthetic methodology used and as a result of these investigations, the following points are noteworthy.

The glycosylation reaction is the critical step in the synthetic pathway and can be achieved directly starting from a commercially available peracetylated sugar donor. Unfortunately this protocol results in only moderate yields and is often complicated by a lack of stereochemical control, which results in difficulty in isolating the desired product. This problem was overcome by the use of trichloroacetimidate (TCA) donors which produce almost exclusively the desired glycan in good yield. The TCA donors are easily prepared by known methods but this results in additional steps for the overall synthesis.

The reduction of the glycosyl nitro compound to the amine initially proved challenging, however using a sodium borohydride/Palladium on carbon reduction protocol has provided a general method to access this class of compound.

The acetyl deprotection was achieved using standard Zemplin deacetylation conditions.

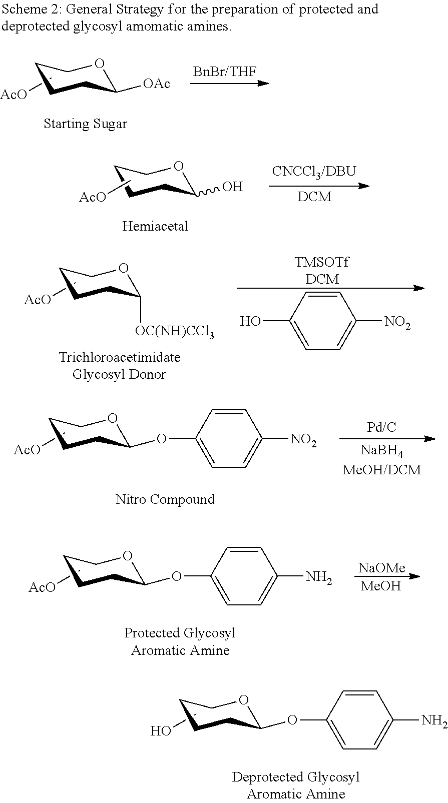

In order to expand the synthetic scope of this methodology and to develop compounds where the carbohydrate unit was removed from the surface material the Peg-triazole linked compound illustrated in FIG. 3 was prepared. The synthetic method for formation of the amine follows the same general procedure as described for the monosaccharides above.

Grafting of Diazonium Salts onto Carbon and Other Materials:

Surface attachment of carbohydrate precursor diazonium compounds has been characterised using Infrared Reflectance Absorption Spectroscopy (IRRAS). Diazotization was carried out in situ using nitrite/tetrafluoroboric acid aqueous solutions containing 100 μM concentration of the precursors. Substrates were reacted for 1 h in the solution, then rinsed with copious amounts of water and sonicated in order to remove weakly bound molecules. An acetyl group was attached to the —OH groups of the precursor in order to serve as reporter group for infrared spectroscopy thanks to the intense carbonyl stretching band. IRRAS experiments were carried out using the galactose-bearing precursor; examples of IRRAS spectra (p-polarization 70-80° incidence; 4 cm⁻¹ resolution, 256 scans) resulting from the attachment procedure follow below. Diagnostic peaks were:

C═O stretching from carbonyl groups, in the region 1700-1760 cm⁻¹;

C—O stretching at 1210-1260 cm⁻¹

C—O stretching 1000-1080 cm⁻¹;

—CH₃ deformations at 1360-1370 cm⁻¹;

C—C skeletal vibration of aromatic rings, typically mode 19a at 1500-1550 cm⁻¹;

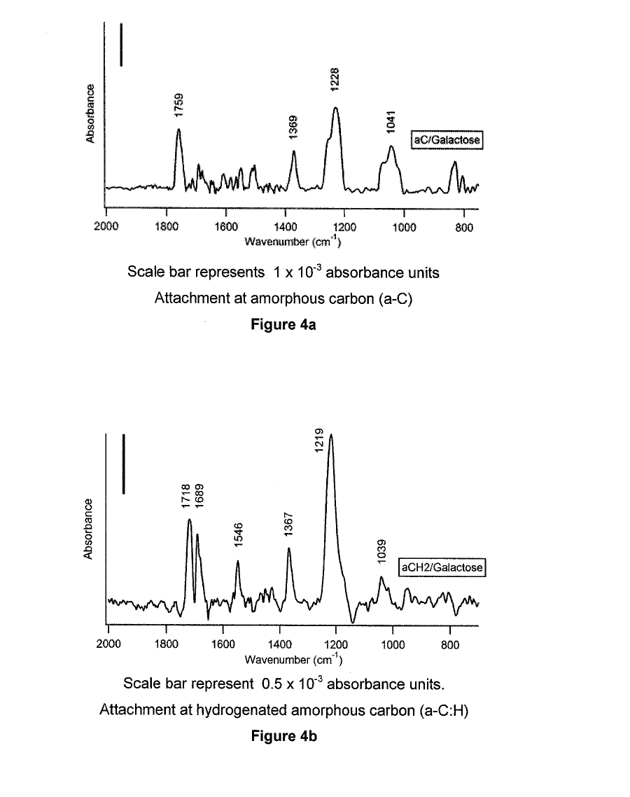

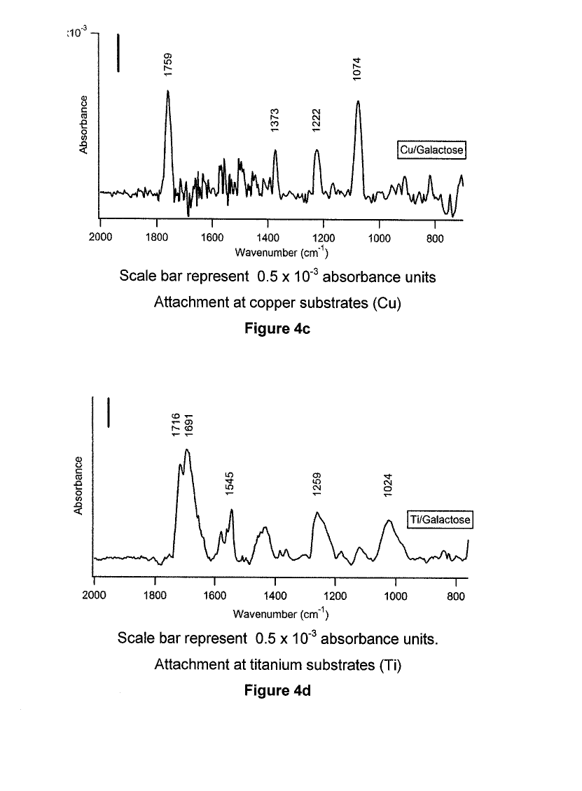

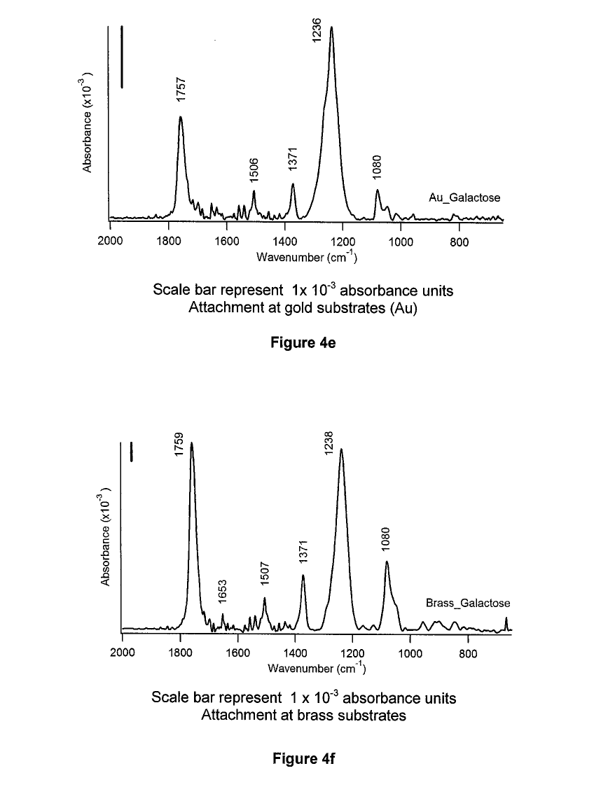

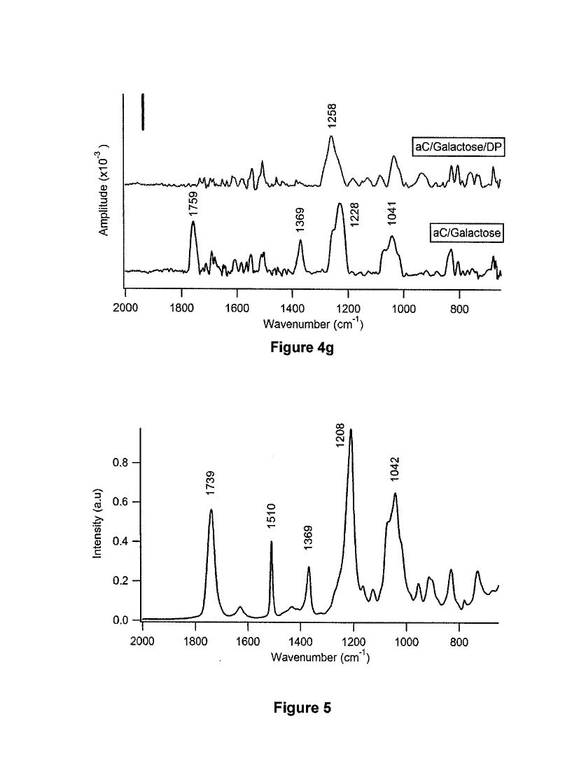

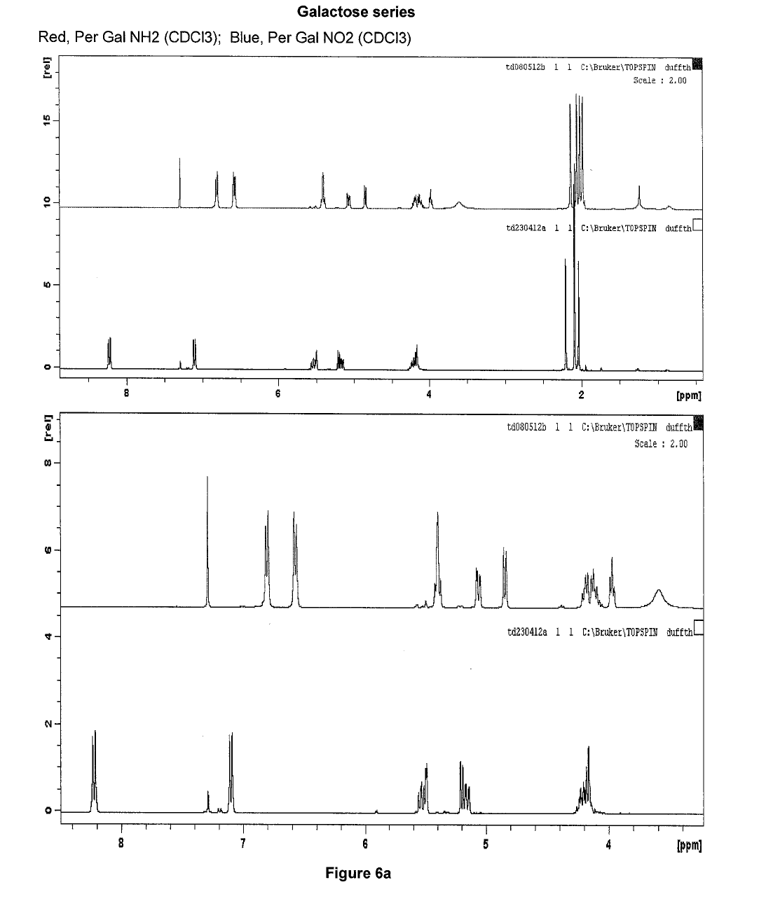

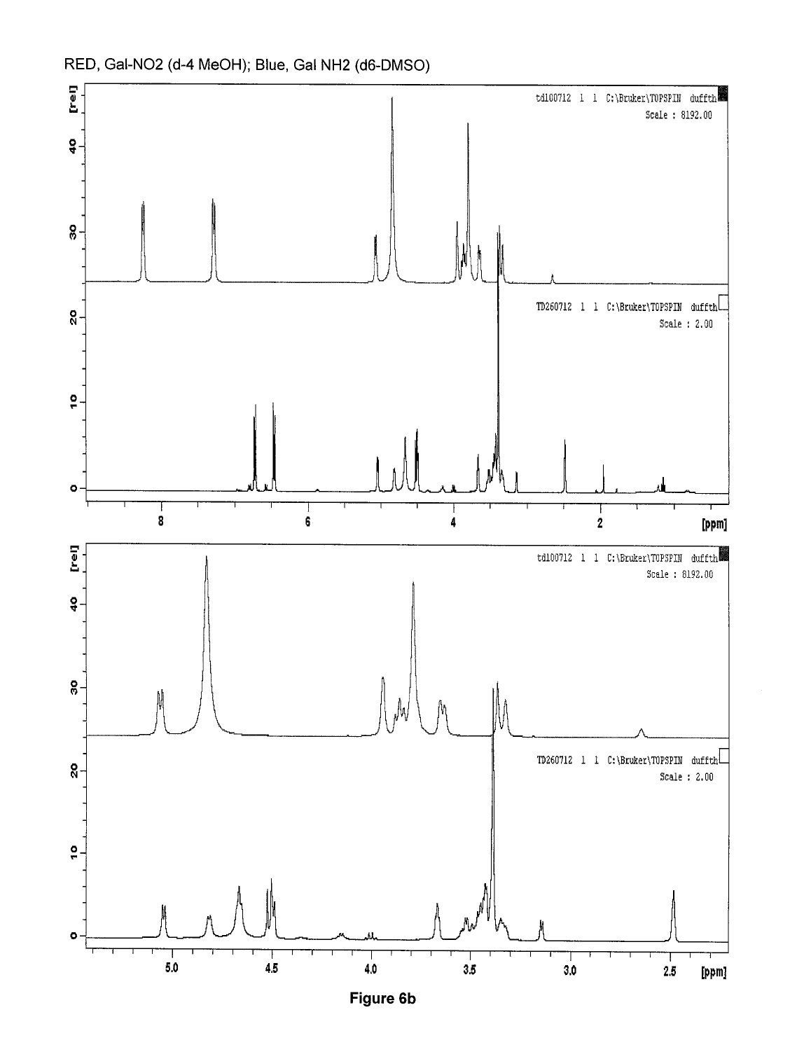

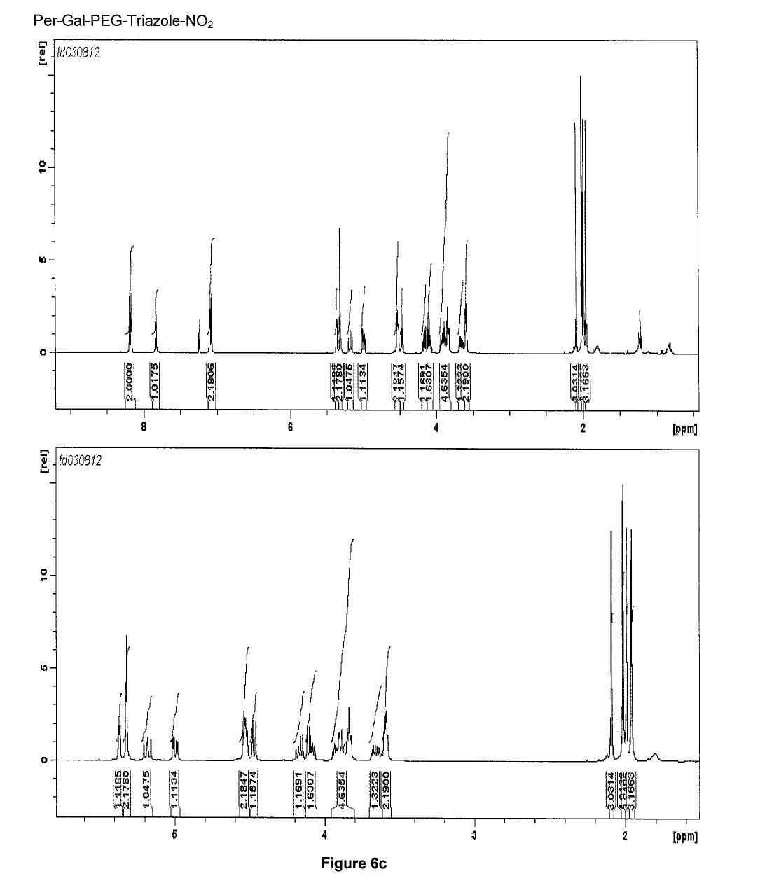

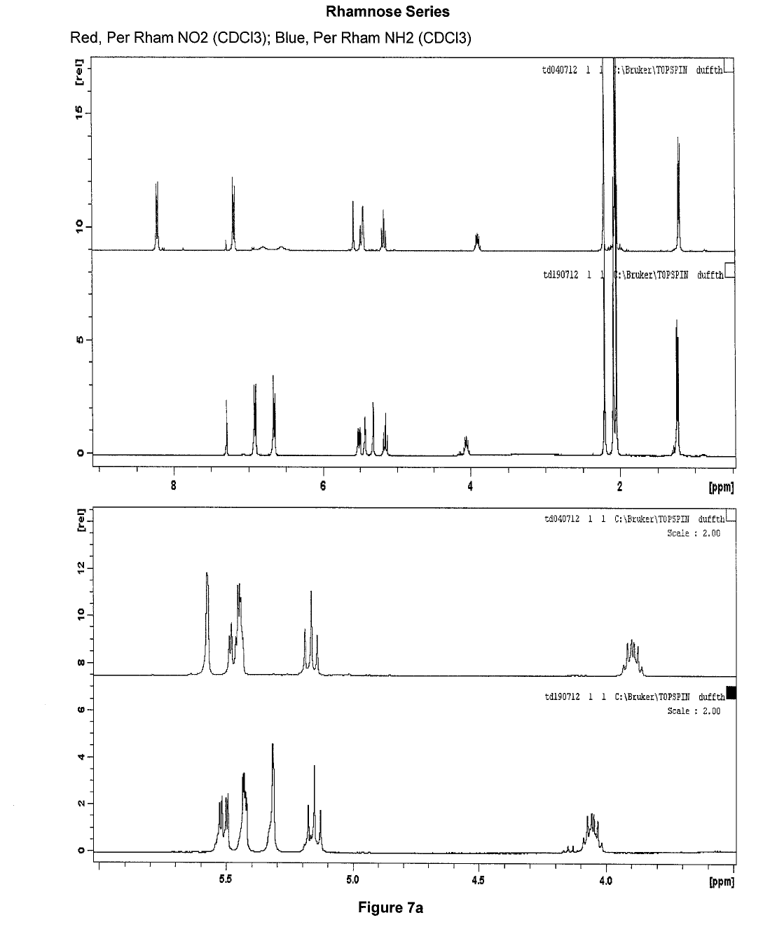

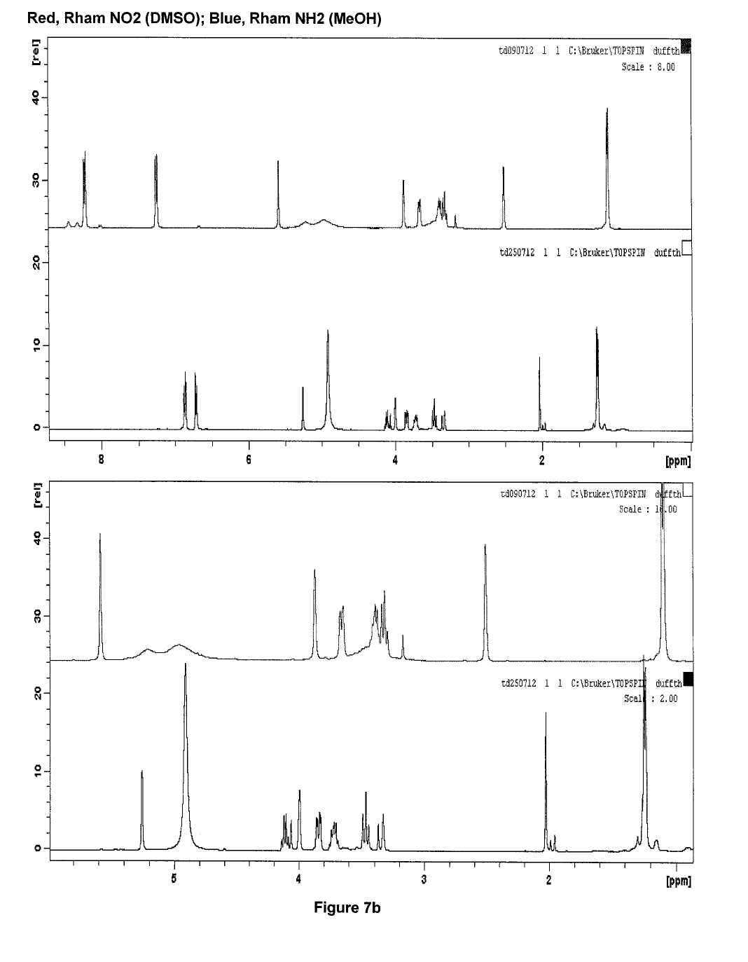

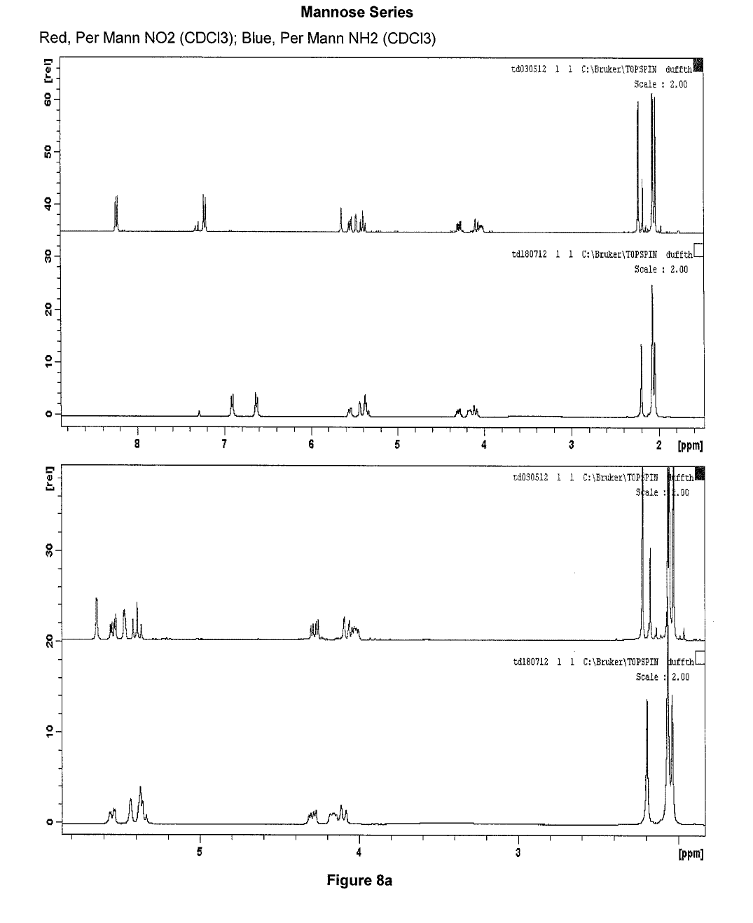

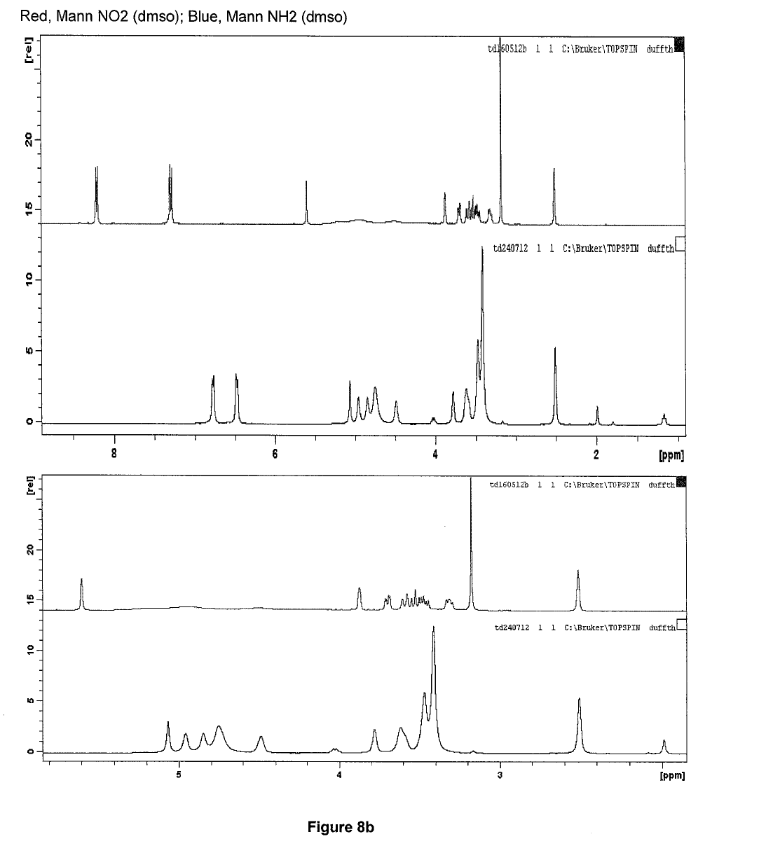

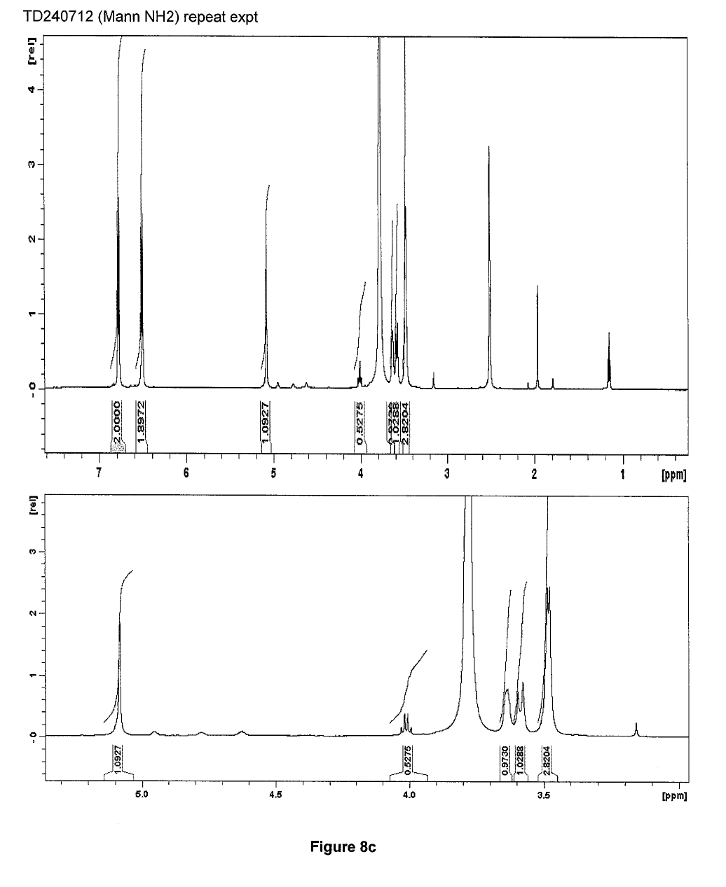

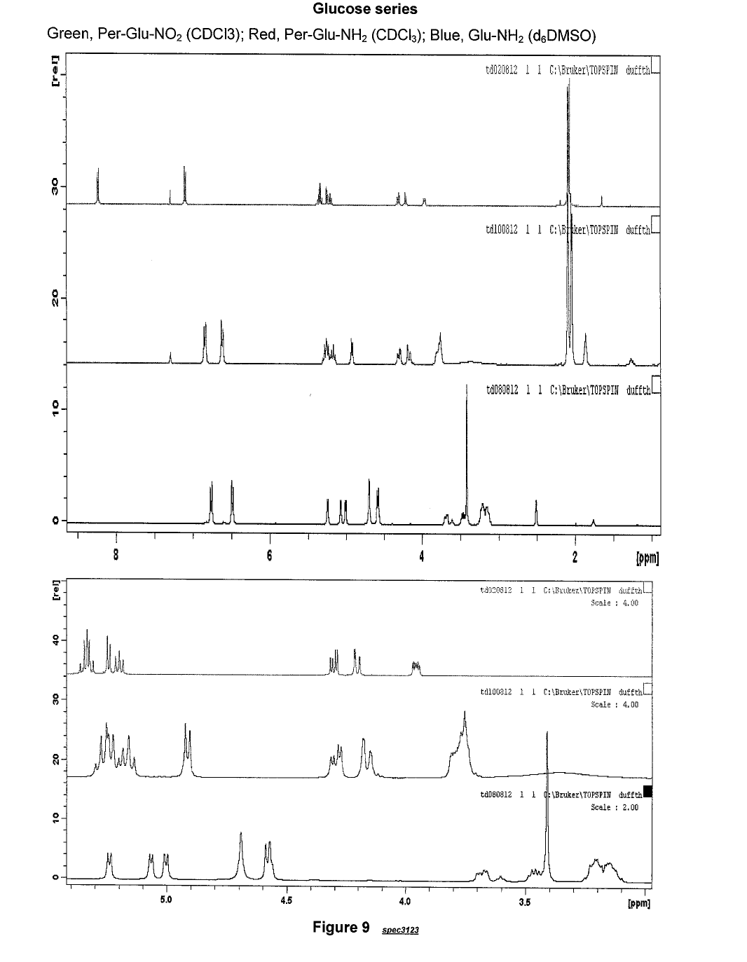

Assignments were based on Infrared and Raman characteristic group frequencies. Tables and Charts by G. Socrates; John Wiley & Sons, Chichester, 2001 (see FIG. 4). IRRAS spectra showed that the carbohydrate precursors displayed characteristic chemistry of these compounds at the surface. For instance the inventors carried out deacetylation reactions using sodium methoxide in methanol. The IRRAS spectra below show how the peaks that are characteristic of the acetyl moiety are lost (C═O stretching, —CH₃ deformation and part of the C—O single bond). The remaining peaks are consistent with the presence of carbohydrates and aromatic rings at the surface (C—O stretchings and C—C skeletal stretching). IRRAS spectra (FIG. 5) showing successful deacetylation at amorphous carbon surfaces. Bottom trace represents the acetylated carbohydrate precursor after attachment at surface; top trace is the spectrum after deacetylation.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A surface having a carbohydrate immobilised thereon,
the carbohydrate having a linker moiety covalently bound thereto, the linker moiety disposed between the surface and the carbohydrate, and
the linker moiety comprising a carbon atom that forms a covalent bond with an atom on the surface, wherein
the carbon atom of the linker moiety that forms a covalent bond with an atom on the surface is not substituted with a ═O, ═N or a ═S moiety,
wherein
the carbon atom in the linker moiety that forms a covalent bond with an atom on the surface is a component of an aromatic or aryl ring.

2. A surface according to claim 1, wherein the carbohydrate is selected from the group consisting of: natural and synthetic monosaccharides, disaccharides, oligosaccharides and polysaccharides.

3. A surface according to claim 1, wherein the surface is selected from the group consisting of diamond like carbon, amorphous carbon, hydrogenated tetrahedral carbon, glassy carbon, vitreous carbon, turbostratic carbon; carbon blacks; single crystal diamond, nanocrystalline diamond, polycrystalline diamond, doped or undoped graphene, doped or undoped polycrystalline graphite, doped or undoped highly ordered graphite, doped or undoped graphite oxide, doped or undoped carbon nanotubes, doped or undoped silicon carbide, doped or undoped titanium carbide, metals including Ti, V, Cr, Mn, Fe, Co, Ni, Cu, Zn, Zr, Nb, Mo, Tc, Ru, Rh, Pd, Ag, Cd, Ta, W, Ir, Pt, Au, Hg, In, Sn, Pb, Al, Bi, Tl, Ga, Si or Ge and alloys thereof containing at least one of said metals, such as stainless steels, brasses, bronzes or nickel alloys (e.g. nitinol, hastelloys); GaAs, ITO (indium tin oxide), tin oxide, SiO₂, titanium oxide, iron oxides, manganese oxides, zinc oxides, polystyrene, polythene, nylon, polytetrafluoroethylene (PTFE), polyestersulfone, polyethyleneterephthalate (PET), polyethersulfone (PES), polyvinyl chlorides (PVC), polystyrenes (PS), polyesters, polyepoxides, polyacetates (e.g. polyvinylacetate), polyethylene oxide, polymethylene oxide, polyphenyl oxide, silicones, polybutadiene, polyacrilonitrile, polypropylene (PP), polyethylene (PE), polyvinylidenefluoride (PVDF), polybutylene (PB), Perfluoroalkoxy (PFA), polytetrafluoroethylene (PTFE), fluorinated ethylene propylene (FEP), ethylene chlorotrifluoroethylene (ECTFE), ethylene trifluoroethylene (ETFE), polycarbonates (PC), polyestersulfone (PES), polysulfones (e.g. polyethersulfone), Polyetheretherketone (PEEK), Polyetherimide (PEI), polyamides (e.g. Nylon, Aramids), polyimides (e.g. Vespel), poly(vinyl alcohol) (PVA), polyacrylics (e.g. PMMA, PAA), polyoxymethylenes (POM), polyurethanes, polyethylene terephthalate (PET), polytrimethylene terephthalate (PTT), polybutyrate, melamine and combinations thereof.

4. A surface according to claim 1, wherein the surface comprises carbon such that the linker moiety is bonded to the surface by means of a C—C bond.

5. A surface according to claim 1, wherein the linker moiety is covalently bound to the carbohydrate by means of a glycosidic bond.

6. A surface according to claim 1, wherein the linker moiety is selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₅-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy.

7. A method of immobilising a carbohydrate to a surface, the method comprising:
i) providing a carbohydrate having a linker moiety covalently bound thereto, wherein the linker moiety comprises a carbon atom bonded to a diazonium cation, and wherein the carbon atom in the linker moiety that is bonded to the diazonium cation is a component of an aromatic or aryl ring such that the diazonium cation is an aryl diazonium cation; and
ii) reacting the diazonium cation with the surface, such that reduction of the diazonium cation results in the carbon atom of the linker moiety forming a covalent bond with an atom on the surface.

8. A method according to claim 7, wherein the carbohydrate is selected from the group consisting of: natural and synthetic monosaccharides, disaccharides, oligosaccharides and polysaccharides.

9. A method according to claim 7, wherein the surface comprises carbon such that the linker moiety is bonded to the surface by means of a C—C bond.

10. A method according to claim 7, wherein the linker moiety is covalently bound to the carbohydrate by means of a glycosidic bond.

11. A method according to claim 7, wherein the linker moiety is selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₃-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy.

12. The method according to claim 7 wherein the carbohydrate is a molecule of the general formula (II) 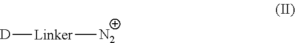
wherein D is a carbohydrate moiety; and
the linker moiety is selected from the group consisting of C₅-C₂₀ aryl, C₃-C₂₀ heteroaryl, C₅-C₂₀ aryloxy, C₃-C₂₀ heteroaryloxy, C₅-C₂₀ aryl substituted with C₁-C₂₀ aliphatic, C₅-C₂₀ aryl substituted with C₃-C₂₀ cycloaliphatic, C₃-C₂₀ heteroaryl substituted with C₁-C₂₀ aliphatic, C₃-C₂₀ heteroaryl substituted with C₃-C₂₀ cycloaliphatic, C₃-C₂₀ heteroaryl substituted with C₁-C₂₀ heteroaliphatic, and C₃-C₂₀ heteroaryl substituted with C₃-C₂₀ cycloheteroaliphatic, wherein each of the above moieties can be optionally substituted one or more times with at least one of hydroxy, Cl, Br, I, F, cyano, C₁-C₅ alkoxy, and C₁-C₅ thioalkoxy,
wherein the diazonium cation is bonded to a carbon atom in the linker moiety, wherein the carbon atom being a component of an aromatic or aryl ring and the diazonium cation being an aryl diazonium cation.

13. A device for in-vivo implantation comprising the surface according to claim 1.

14. A diagnostic kit comprising the surface according to claim 1.

15. An article of manufacture comprising the surface according to claim 1, wherein said article is selected from the group consisting of
a material for in vivo implantation;
a bio-fouling resistant material;
a scaffold for tissue culture and engineering;
a screening array for monitoring glycoprotein interactions;
a diagnostic kit;
a drug delivery vehicle; or
a chromatographic stationary phase; equipment/materials involved in food manufacture, processing or dispensing;
a sensor such as aquatic, bioanalytical, electrochemical;
surgical/veterinary utensils; and a tool for agriculture and livestock.

16. An application comprising the surface according to claim 1, wherein said application is selected from the groups consisting of: drug delivery; antifouling coatings; glycoarrays; filtration membranes; biomedical devices such as implants, sensors, catheters, guidewires, dental parts; equipment/materials involved in food manufacture, processing or dispensing; sensors such as aquatic, bioanalytical, electrochemical; solid phases for separation/filtration; surgical/veterinary utensils; and tools for agriculture and livestock.
